(19) United States
(12) Patent Application Publication
(10) Pub. No.: US 2025/0352705 A1
(43) Pub. Date: Nov. 20, 2025

(54) SORBENT CARTRIDGE DESIGNS

(71) Applicant: Mozarc Medical US LLC, Minneapolis, MN (US)

(72) Inventors: Bryant J. Pudil, Plymouth, MN (US); Mark David Schneider, Mound, MN (US); Michael J. Elvidge, Minneapolis, MN (US)

(21) Appl. No.: 19/280,939

(22) Filed: Jul. 25, 2025

Related U.S. Application Data
(63) Continuation of application No. 17/323,081, filed on May 18, 2021, now Pat. No. 12,397,093.

(51) Int. Cl. A61M 1/16; A61M 39/10; B01D 15/18; B01D 15/22; B01J 20/02; B01J 20/06; B01J 20/08; B01J 20/20; B01J 20/24; B01J 20/28; B01J 39/12; B01J 41/10

(52) U.S. Cl. CPC A61M 1/1609; A61M 1/1656; A61M 39/10; B01D 15/18; B01D 15/22; B01J 20/0211; B01J 20/06; B01J 20/08; B01J 20/20; B01J 20/24; B01J 20/28045; B01J 39/12; B01J 41/10; A61M 2205/3334; B01J 2220/62

(57) ABSTRACT

Sorbent cartridges having a flow control insert to improve the functional capacity of a sorbent cartridge is provided. Flow control inserts can include a plurality of flow channels filled with sorbent material through which fluid to be regenerated can travel in the sorbent cartridge.

### CROSS-REFERENCE TO RELATED APPLICATIONS

This application is a continuation of U.S. patent application Ser. No. 17/323,081, filed May 18, 2021 and entitled “SORBENT CARTRIDGE DESIGNS,” the entirety of which is herein incorporated by reference.

### FIELD

The disclosure relates to sorbent cartridges having a flow control insert to improve the functional capacity of the sorbent cartridge. The flow control insert can include a plurality of flow channels filled with sorbent material through which fluid to be regenerated can travel in the sorbent cartridge.

### BACKGROUND

Sorbent-based multi-pass dialysis systems can reduce the volume of purified water needed for therapy. Sorbent cartridges operate by adsorbing ions and other waste solutes from spent dialysate, allowing the repurified to be reused. However, sorbent materials have a finite capacity. If the capacity of a sorbent cartridge is reached during treatment, treatment must be stopped. Due to channeling and non-uniform flow of dialysate through a sorbent cartridge, certain portions of the sorbent material may undergo greater ion exchange with the dialysate than others, increasing the rate at which the capacity of these portions of sorbent material is reached, which allows solutes intended to be removed by the sorbent cartridge to return to the dialyzer.

Hence, there is a need for systems and methods that can increase the adsorptive capacity of a sorbent cartridge, allowing full treatment of larger or more uremic patients without increasing the size requirements for the sorbent cartridge. There is a need for systems and methods that can improve flow distribution of dialysate through the sorbent cartridge, allowing more effective solute removal by the sorbent cartridge.

### SUMMARY OF THE INVENTION

The problem to be solved is increasing the adsorptive capacity of a sorbent cartridge. The solution is to include a flow control insert within the sorbent cartridge to more efficiently distribute fluid flow throughout the sorbent material.

The first aspect relates to a sorbent cartridge. In any embodiment, the sorbent cartridge can include a sorbent cartridge casing; at least one sorbent material inside the sorbent casing; and a flow control insert; the flow control insert having a plurality of flow channels filled with the at least one sorbent material.

In any embodiment, the at least one sorbent material can include zirconium phosphate.

In any embodiment, the at least one sorbent material can include zirconium oxide.

In any embodiment, the at least one sorbent material can include at least one sorbent material selected from a group of activated carbon, alumina, urease, an anion exchange material, a cation exchange material, zirconium phosphate, and zirconium oxide.

In any embodiment, each of the plurality of flow channels can have a hexagonal shape.

In any embodiment, the plurality of flow channels can form a plurality of cylinders.

In any embodiment, the plurality of cylinders can be a plurality of concentric cylinders.

In any embodiment, the flow control insert can have a substantially flat top portion.

In any embodiment, the flow control insert can have a dome-shaped top portion.

In any embodiment, the sorbent cartridge can include a compression insert between a top portion of the flow control insert and an inner top of the sorbent cartridge casing.

In any embodiment, the flow control insert can contact the compression insert.

The features disclosed as being part of the first aspect can be in the first aspect, either alone or in combination, or follow any arrangement or permutation of any one or more of the described elements. Similarly, any features disclosed as being part of the first aspect can be in a second or third aspect described below, either alone or in combination, or follow any arrangement or permutation of any one or more of the described elements.

The second aspect relates to a flow control insert for a sorbent cartridge. In any embodiment, the flow control insert can include a plurality of flow channels; the flow control insert insertable into a sorbent cartridge containing at least one sorbent material.

In any embodiment, each of the plurality of flow channels can have a substantially hexagon shape.

In any embodiment, the plurality of flow channels can form a plurality of cylinders.

In any embodiment, the plurality of cylinders can be a plurality of concentric cylinders.

In any embodiment, the flow control insert can have a substantially flat top portion.

In any embodiment, the flow control insert can have a dome-shaped top portion.

In any embodiment, the flow control insert can be constructed from a material selected from: polypropylene, polyethylene, stainless steel, glass, plastic, or a combination thereof.

The features disclosed as being part of the second aspect can be in the second aspect, either alone or in combination, or follow any arrangement or permutation of any one or more of the described elements. Similarly, any features disclosed as being part of the second aspect can be in the first or third aspect, either alone or in combination, or follow any arrangement or permutation of any one or more of the described elements.

The third aspect is drawn to a dialysate flow path. In any embodiment, the dialysate flow path can include a first connector fluidly connectable to a dialyzer outlet; a second connector fluidly connectable to a dialyzer inlet; at least one dialysate pump; and at least a first sorbent module; the sorbent module including: a sorbent cartridge casing; at least one sorbent material inside the sorbent casing; and a flow control insert; the flow control insert having a plurality of flow channels filled with the at least one sorbent material.

In any embodiment, the dialysate flow path can include at least a second sorbent module; wherein the second sorbent module has a second sorbent cartridge casing; at least one sorbent material inside the second sorbent casing; and a second flow control insert; the second flow control insert having a plurality of flow channels filed with the at least one sorbent material.

In any embodiment, the first and second sorbent modules can contain different sorbent materials.

In any embodiment, the first sorbent module can contain at least one sorbent material selected from the group of: activated carbon, alumina, urease, zirconium phosphate, zirconium oxide, an anion exchange material, and a cation exchange material.

The features disclosed as being part of the third aspect can be in the third aspect, either alone or in combination, or follow any arrangement or permutation of any one or more of the described elements. Similarly, any features disclosed as being part of the third aspect can be in the first or second aspect, either alone or in combination, or follow any arrangement or permutation of any one or more of the described elements.

### BRIEF DESCRIPTION OF THE DRAWINGS

FIGS. 1A-C illustrate a flow control insert having small hexagonal flow channels.

FIGS. 2A-C illustrate a flow control insert having larger hexagonal flow channels.

FIGS. 3A-C illustrate a flow control insert having medium sized hexagonal flow channels.

FIGS. 4A-C illustrate a flow control insert having large sized hexagonal flow channels.

FIGS. 5A-C illustrate a flow control insert having flow channels forming concentric cylinders.

FIG. 6 illustrates a flow control insert having a substantially flat top portion.

FIG. 7 is a graph showing ammonium ion concentration in sorbent cartridge effluent as a function of volume.

FIG. 8 is a graph showing time averaged ammonium ion concentration in sorbent cartridge effluent as a function of volume.

FIG. 9 is a graph showing potassium ion concentration in sorbent cartridge effluent as a function of volume.

FIG. 10 is a dialysate flow path.

### DETAILED DESCRIPTION

Unless defined otherwise, all technical and scientific terms used have the same meaning as commonly understood by one of ordinary skill in the art.

The articles “a” and “an” are used to refer to one to over one (i.e., to at least one) of the grammatical object of the article. For example, “an element” means one element or over one element.

“Activated carbon” refers to a form of carbon processed to have small pores, increasing the surface area available for adsorption.

“Alumina” refers to aluminum oxide, Al₂O₃.

An “anion exchange material” is a sorbent material that removes anions from solution, replacing the removed anions with different anions.

A “cation exchange material” is a sorbent material that removes cations from solution, replacing the removed cations with different cations.

A “compression insert” is a component that can be deformed, wherein the component holds in place one or more other components when a force is applied.

The term “comprising” includes, but is not limited to, whatever follows the word “comprising.” Use of the term indicates the listed elements are required or mandatory but that other elements are optional and may be present.

The term “concentric cylinders” refers to two or more cylinders having the same center point.

The term “connector” refers to a conduit or component allowing for the passage of fluid or gas.

The term “consisting of” includes and is limited to whatever follows the phrase “consisting of.” The phrase indicates the limited elements are required or mandatory and that no other elements may be present.

The term “consisting essentially of” includes whatever follows the term “consisting essentially of” and additional elements, structures, acts, or features that do not affect the basic operation of the apparatus, structure or method described.

The term “contact” refers to two or more components physically touching each other.

A “cylinder” is a three-dimensional shape having two circular ends with substantially the same diameter.

The term “dialysate flow path” refers to a pathway through which dialysate travels during dialysis therapy.

The term “dialysate pump” refers to a component that can move fluid through a dialysate flow path by applying suction or pressure.

The term “dialyzer” refers to a cartridge or container with two flow paths separated by semi-permeable membranes. One flow path is for blood and one flow path is for dialysate. The membranes can be in hollow fibers, flat sheets, or spiral wound or other conventional forms known to those of skill in the art. Membranes can be selected from the following materials of polysulfone, polyethersulfone, poly (methyl methacrylate), modified cellulose, or other materials known to those skilled in the art.

The term “dialyzer inlet” refers to a connector through which fluid can enter a dialyzer.

The term “dialyzer outlet” refers to a connector through which fluid can exit a dialyzer.

The term “dome-shaped” refers to a top portion of a component that is rounded, with the top portion being higher at the center than around the edges.

A “flow channel” is a conduit or pathway through a flow control insert through which fluid can travel.

A “flow control insert” is a component inside of a sorbent cartridge that directs the flow of fluid through the sorbent cartridge.

The term “fluidly connectable” refers to the ability to provide passage of fluid, gas, or combinations thereof, from one point to another point. The ability to provide such passage can be any mechanical connection, fastening, or forming between two points to permit the flow of fluid, gas, or combinations thereof. The two points can be within or between any one or more of compartments, modules, systems, components, and rechargers, all of any type. Notably, the components that are fluidly connectable, need not be a part of a structure. For example, an outlet “fluidly connectable” to a pump does not require the pump, but merely that the outlet has the features necessary for fluid connection to the pump.

The term “fluidly connected” refers to a particular state or configuration of one or more components such that fluid, gas, or combination thereof, can flow from one point to another point. The connection state can also include an optional unconnected state or configuration, such that the two points are disconnected from each other to discontinue flow. It will be further understood that the two “fluidly connectable” points, as defined above, can form a “fluidly connected” state. The two points can be within or between any one or more of compartments, modules, systems, components, all of any type.

The term “glass” refers to a mixture of silicates forming a hard material.

The term “hexagonal” or a “hexagonal shape” refers to a two-dimensional shape having six sides.

The term “inner top” refers to the top portion of a container or component inside of the container or component.

“Plastic” refers to a class of synthetic materials made from organic polymers.

The term “plurality” can mean any one or more of a component or a thing.

The term “polyethylene” refers to a synthetic polymer of ethylene.

The term “polypropylene” refers to a synthetic polymer of propylene.

The terms “sorbent cartridge” and “sorbent container” can refer to a cartridge containing one or more sorbent materials for removing specific solutes from solution, such as urea. The term “sorbent cartridge” does not require the contents in the cartridge be sorbent based, and the contents of the sorbent cartridge can be any contents that can remove waste products from a dialysate. The sorbent cartridge may include any suitable amount of one or more sorbent materials. In certain instances, the term “sorbent cartridge” can refer to a cartridge which includes one or more sorbent materials in addition to one or more other materials capable of removing waste products from dialysate. “Sorbent cartridge” can include configurations where at least some materials in the cartridge do not act by mechanisms of adsorption or absorption. In any embodiment, a system may include a number of separate cartridges which can be physically separated or interconnected wherein such cartridges can be optionally detached and reattached as desired.

The term “sorbent cartridge casing” refers to the walls forming a sorbent cartridge inside which sorbent material is placed.

“Sorbent materials” are materials capable of removing specific solutes from solution, such as cations or anions.

A “sorbent module” means a discreet component of a sorbent cartridge. Multiple sorbent cartridge modules can be fitted together to form a sorbent cartridge of two, three, or more sorbent cartridge modules. The “sorbent cartridge module” or “sorbent module” can contain any selected materials for use in sorbent dialysis and may or may not contain a “sorbent material” or adsorbent, but less than the full complement of sorbent materials needed. In other words, the “sorbent cartridge module” or “sorbent module” generally refers to the use of the “sorbent cartridge module” or “sorbent module” in sorbent-based dialysis, e.g., REDY (Recirculating DYalysis), and not that a “sorbent material” that is necessarily contained in the “sorbent cartridge module” or “sorbent module.”

“Stainless steel” refers to steel containing chromium and is resistant to rusting.

The term “substantially flat” refers to a surface or layer of a component wherein the top of the surface or layer is at essentially the same elevation when the component is positioned for normal use.

The term “top portion” refers to a part of a component that is intended to be at a higher elevation when the component is positioned for normal use.

“Urease” is an enzyme that converts urea to ammonium ions and carbon dioxide.

“Zirconium oxide” is a sorbent material that removes anions from a fluid, exchanging the removed anions for different anions. Zirconium oxide can also be formed as hydrous zirconium oxide.

“Zirconium phosphate” is a sorbent material that removes cations from a fluid, exchanging the removed cations for different cations.

### Sorbent Cartridge Design

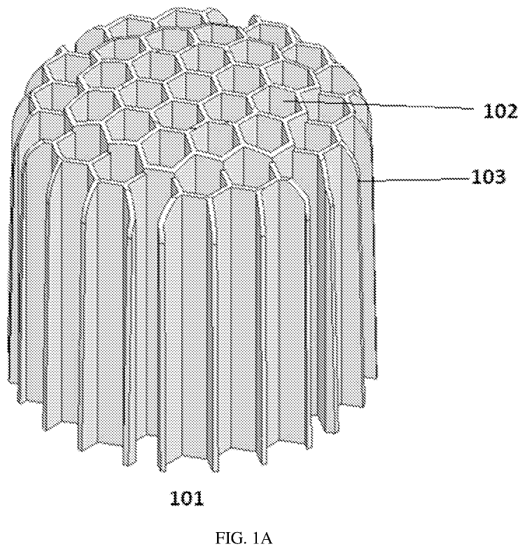

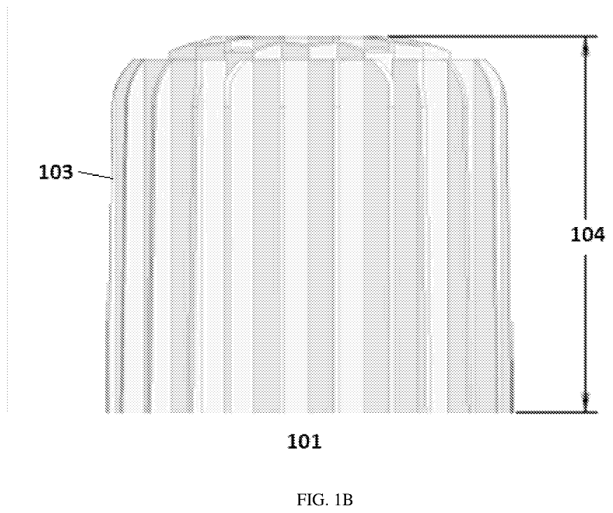

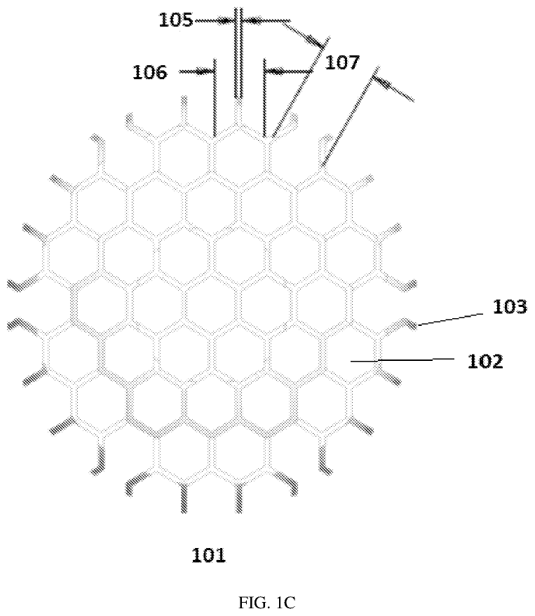

FIGS. 1A-C illustrate a flow control insert 101 for a sorbent cartridge resulting in improved flow distribution through the sorbent cartridge and more efficient removal of solutes from dialysate. FIG. 1A is a perspective view of the flow control insert 101, FIG. 1B is a side view of the flow control insert 101 and FIG. 1C is a schematic of a top view of the flow control insert 101. The flow control insert 101 can fit into a casing of a sorbent cartridge (not shown). During use, spent dialysate can travel through the sorbent cartridge, and the flow control insert will improve fluid flow distribution through the sorbent cartridge.

As illustrated in FIG. 1A, the flow control insert 101 includes a plurality of flow channels 102. For clarity, only one of the flow channels is labeled 102. In certain embodiments, as illustrated in FIG. 1A, each of the flow channels 102 can have a substantially hexagonal shape, forming a honey-comb type structure. The hexagonal shape of the flow channels 102 mimics a cylinder, which provides better flow distribution than many other shapes while minimizing space inside the sorbent cartridge lost to the flow control insert 101. The flow control insert 101 can contact an inside of a sorbent cartridge casing at ends 103. The flow control insert 101 can be held in place by a friction fit between the ends 103 and the sorbent cartridge casing, as well as by the addition of sorbent material and a seal between the top of the sorbent cartridge casing and the sorbent material. In certain embodiments, a foam compression insert can be included between the top portion of the flow control insert and an inner top of the sorbent cartridge casing to form a compression fit when the foam compression insert is compressed. The sorbent cartridge having the flow control insert 101 can contain any type of sorbent material. As a non-limiting example, the sorbent cartridge can contain a cation exchange material, such as zirconium phosphate, for removal of cations. The sorbent cartridge can also contain an anion exchange material, such as zirconium oxide, for removal of anions. Additional materials can include urease for conversion of urea to ammonium ions, alumina for use as a urease support, and/or activated carbon for removal of non-ionic solutes. Other ion exchange resins or materials can be used. The sorbent cartridge can contain any one or more sorbent materials, either arranged as discrete layers in the sorbent cartridge or mixed together.

As illustrated in in FIG. 1B, in certain embodiments, the flow control insert 101 can have a dome-shaped top. However, as described, the flow control insert 101 can alternatively have a substantially flat top portion. The height of the flow control insert 101, shown as distance 104, can be varied depending on the size of the sorbent cartridge to be used. In certain embodiments, the flow control insert 101 can be used with a filter pad or other component between the flow control insert 101 and the top and/or bottom of the sorbent cartridge. If a filter pad or other component is to be placed between the flow control insert 101 and the top and/or bottom of the sorbent cartridge, the height of the flow control insert 101 can be smaller than the height of the sorbent cartridge.

As illustrated in FIG. 1C, the diameter 107 of each hexagon flow channel 102 can be a function of the diameter of the flow control insert 101 and the number of hexagonal flow channels 102 included. One of skill in the art will be able to calculate the diameter 107, as well as the distance 106 between each point, based on the number of hexagonal flow channels 102 included and the diameter of the flow control insert 101. The width 105 of the solid portions of flow control insert 101 can be minimized to reduce the volume of space taken up by the flow control insert 101 inside the sorbent cartridge. However, the width 105 should be thick enough to provide stability to the flow control insert 101. The width 105 can be varied according to the needs of the system or user and the materials used, and can range from about 0.1 mm to about 4 mm. In certain embodiments, the width 105 can be about 2 mm.

The flow control insert 101 can be made from any material that is stable to the conditions in which the sorbent cartridge is to be used. Polypropylene, polyethylene, stainless steel, glass, or other plastics can be used.

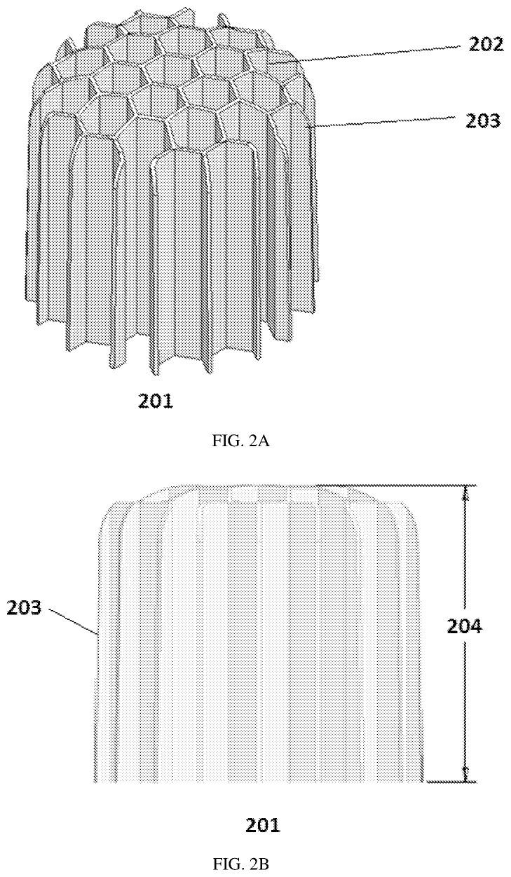

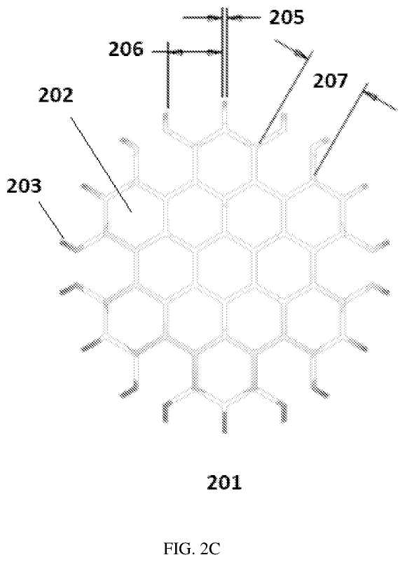

FIGS. 2A-C illustrate a flow control insert 201 having larger hexagonal flow channels 202 than those illustrated in FIGS. 1A-C. FIG. 2A is a perspective view of the flow control insert 201, FIG. 2B is a side view of the flow control insert 201 and FIG. 2C is a schematic of a top view of the flow control insert 201.

As illustrated in FIG. 2A, the flow control insert 201 includes a plurality of hexagonal flow channels 202, only one of which is labeled in FIGS. 2A-C. The flow control insert 201 can contact an inside of a sorbent cartridge casing at ends 203. The larger flow channels 202 as compared to those in FIGS. 1A-C, result in a smaller volume for the flow control insert 201 and therefore a lower size requirement for the sorbent cartridge. However, the larger flow channels 202 result in a slightly less effective flow control insert 201 at distributing flow throughout the sorbent cartridge.

As illustrated in in FIG. 2B, similar to the flow control insert of FIGS. 1A-C, the flow control insert 201 can have a dome-shaped top portion. However, as described, any described flow control insert 201 can alternatively have a substantially flat top portion. The height of the flow control insert 201, shown as distance 204, can be varied depending on the size of the sorbent cartridge to be used and is not dependent on the size of each flow channel 202.

As illustrated in FIG. 2C, the diameter 207 of each hexagon flow channel 202 depends on the diameter of the flow control insert 201 and the number of hexagonal flow channels 202 included. As such, the diameter 207 of each flow channel 202 is larger than the diameters used in the flow control insert of FIGS. IA-C. One of skill in the art will be able to calculate the diameter 207, as well as the distance 206 between each point, based on the number of hexagonal flow channels 202 included and the diameter of the flow control insert 201. The width 205 of the solid portions of flow control insert 201 does not depend on the number of flow channels 202, and can be the same as described with reference to FIGS. 1A-C.

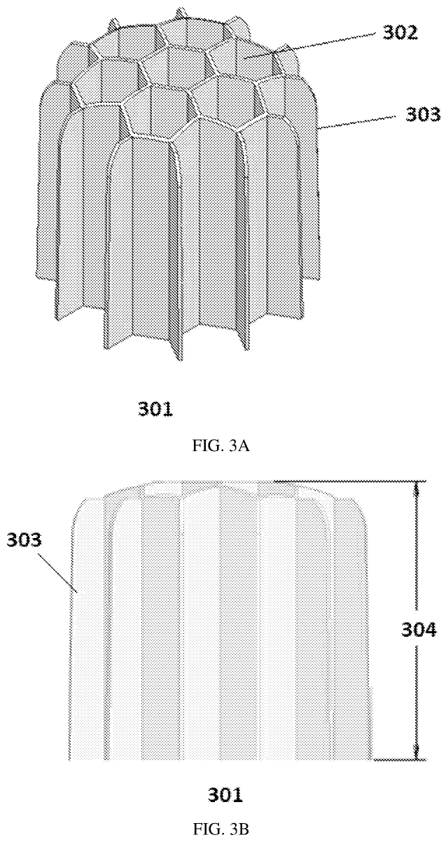

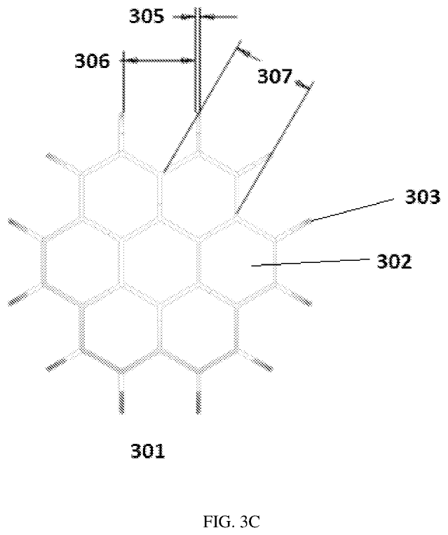

FIGS. 3A-C illustrate a flow control insert 301 having still larger flow channels 302. FIG. 3A is a perspective view of the flow control insert 301, FIG. 3B is a side view of the flow control insert 301 and FIG. 3C is a schematic of a top view of the flow control insert 301. Similar to the flow control inserts of FIGS. 1-2, the flow control insert 301 of FIG. 3A can contact an inside of a sorbent cartridge casing at ends 303. The larger flow channels 302, result in fewer total flow channels 302, and a smaller volume for the flow control insert 301 and therefore a lower size requirement for the sorbent cartridge. As illustrated in in FIG. 3B, similar to the flow control inserts of FIGS. 1-2, the flow control insert 301 can have a dome-shaped top portion. However, as described, any described flow control insert 301 can alternatively have a substantially flat top portion. The height of the flow control insert 301, shown as distance 304, can be varied depending on the size of the sorbent cartridge to be used and is not dependent on the size of each flow channel 302. For example, if the inner portion of the sorbent cartridge is 135 mm high, the flow control insert can be between 30 mm and 135 mm in length or between 20% to I 00% of the total inner portion height of the sorbent cartridge. Leaving a space, between 0% to 20% of the total inner portion height of the sorbent cartridge, at the top and/or the bottom of the sorbent cartridge may improve flow through the flow control insert 301 in either flow direction, improving the ability to recharge the sorbent material in the sorbent cartridge without removing the flow control insert 301.

As illustrated in FIG. 3C, the diameter 307 of each hexagon flow channel 302 is larger than that illustrated in FIGS. 1-2. One of skill in the art will be able to calculate the diameter 307, as well as the distance 306 between each point, based on the number of hexagonal flow channels 302 included and the diameter of the flow control insert 301. The width 305 of the solid portions of flow control insert 301 does not depend on the number of flow channels 302.

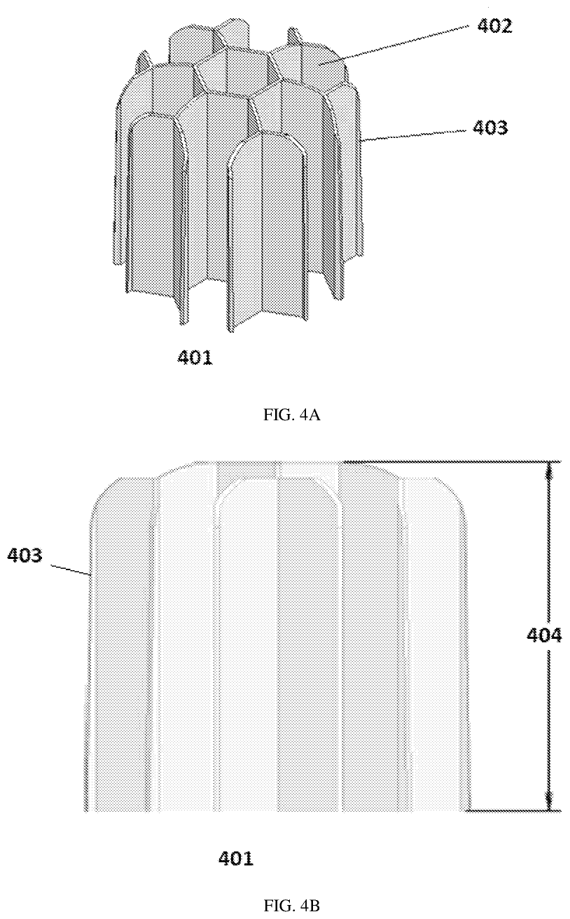

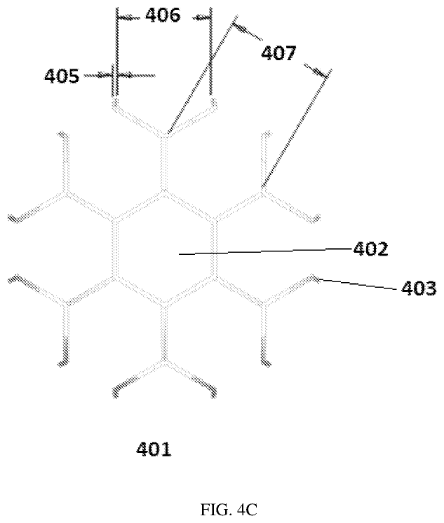

FIGS. 4A-C illustrate a flow control insert 401 having 7 flow channels 402. FIG. 4A is a perspective view of the flow control insert 401, FIG. 4B is a side view of the flow control insert 401 and FIG. 4C is a schematic of a top view of the flow control insert 401. As illustrated in FIG. 3A, the flow control insert 401 can contact an inside of a sorbent cartridge casing at ends 403, forming six of the seven total flow channels 402. As illustrated in in FIG. 4B, similar to the flow control inserts of FIGS. 1-3, the flow control insert 401 can have a dome-shaped top. The height of the flow control insert 401, shown as distance 404, depends on the size of the sorbent cartridge to be used and is not dependent on the size of each flow channel 402. The diameter 407 of each hexagon flow channel 402 as well as the distance 406 between each point larger than that illustrated in FIGS. 1-3. The width 405 of the solid portions of flow control insert 401 can be set based on the needs of the user for stability, durability and to minimize volume of the flow control insert 401.

FIGS. 1-4 each illustrate flow control inserts having hexagonal flow channels with varying sizes. In certain embodiments, the diameters of the flow channels can be between 10 mm to 60 mm.

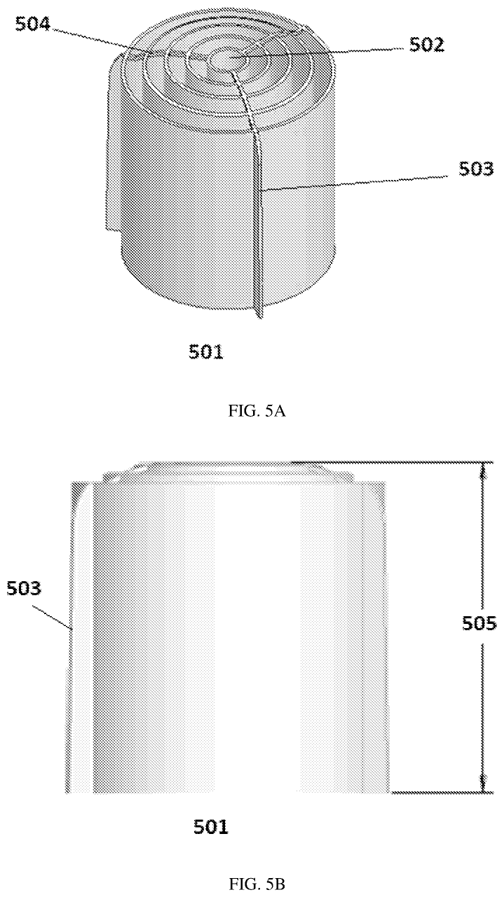

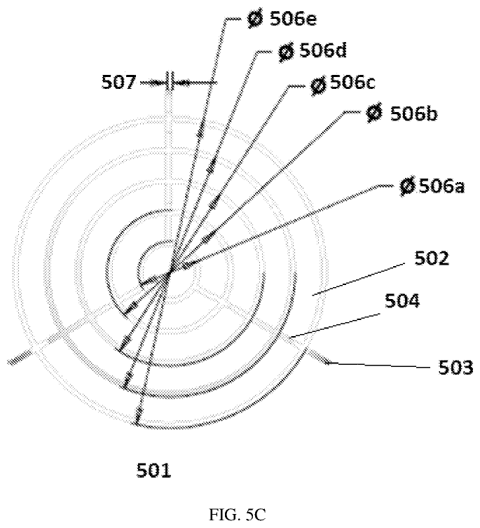

FIGS. 5A-C illustrate a flow control insert 501 having concentric cylinder flow channels 502. FIG. 5A is a perspective view of the flow control insert 101, FIG. 5B is a side view of the flow control insert 501 and FIG. 5C is a schematic of a top view of the flow control insert 501. Similar to the hexagonal flow channel inserts of FIGS. 1-4, the flow control insert 501 of FIGS. 5A-C can fit within a sorbent cartridge (not shown). Concentric cylinders mimic cylindrical flow, improving flow distribution through the sorbent cartridge, similar to the hexagon shaped flow channels illustrated in FIGS. 1-4.

The flow control insert 501 illustrated in FIG. 5A has six concentric flow channels 502, only one of which is labeled for clarity. Although illustrated with six concentric cylinder flow channels 502, the flow control insert 501 can have more or fewer flow channels 502, similar to the designs shown in FIGS. 1-4. The flow control insert 501 can contact an inside of a sorbent cartridge (not shown) casing at ends 503, which form the sixth flow channel 502 between the outside of the flow control insert 501 and the inner wall of the sorbent cartridge casing. Connectors 504 can connect the solid portions of the flow control insert 501 to prevent the concentric cylinder flow channels 502 from expanding or contracting.

As illustrated in in FIG. 5B, the flow control insert 501 can have a dome-shaped top. However, as described, the cylindrical flow control insert 501 can alternatively have a substantially flat top portion, similar to the hexagonal flow control inserts of FIGS. 1-4. The height of the flow control insert 501, shown as distance 505, does not depend on the shape of the flow channels 502 and can be set depending on the size of the sorbent cartridge to be used.

As illustrated in FIG. 5C, the width 507 of the solid portions of flow control insert 501 can be minimized to reduce the volume of space taken up by the flow control insert 501 inside the sorbent cartridge. However, similar to the flow control inserts of FIGS. 1-4, the width 507 should be thick enough to provide stability to the flow control insert 501. The width 507 can be set based on the needs of the user, similar to the flow control inserts of FIGS. 1-4. In certain embodiments, the width 507 can be about 2 mm. In FIG. 5C, 506a is the radius of the inner cylinder, 506b is the radius of the second cylinder, 506c is the radius of the third cylinder, 506d is the radius of the fourth cylinder, and 506e is the radius of the outer cylinder. As with the hexagonal flow channels of FIGS. 1-4, the number of cylindrical flow channels can be varied, and can include 2, 3, 4, 5, 6, 7, or more concentric cylinders depending on the size of the flow control insert 501, the thickness of the flow control insert 501 and the thickness of each concentric cylinder used.

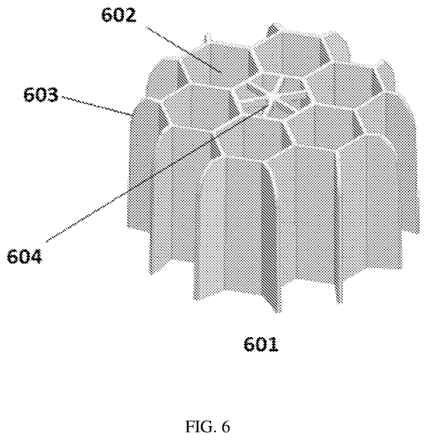

FIG. 6 illustrates a flow control insert 601 having a substantially flat top portion. The flow control insert 601 is similar to the domed flow control insert illustrated in FIGS. 3AC. However, any of the flow control inserts with a dome-shaped top portion illustrated in FIGS. 1-5 can be constructed with a substantially flat top portion. Similar to the other designs, the flow control insert 601 includes a plurality of flow channels 602. Although illustrated as having hexagonal flow channels 602, the flat flow control insert 601 can alternatively have concentric cylinder flow channels, similar to the design illustrated in FIGS. 5A-C. The flow control insert 601 can be placed in a sorbent cartridge (not shown), with the end portions 603 contacting the wall of the sorbent cartridge casing, forming the outer flow channels. In certain embodiments, spokes 604 can be included in the center flow channel 602 to improve stability and manufacturability.

The top of the dome-shaped flow control inserts illustrated in FIGS. 1-5 can conform to the shape of the sorbent cartridge casing, while the substantially flat flow control insert of FIG. 6 can leave a small space between the top of the flow control insert and the top of the sorbent cartridge. The additional space formed with the flat top flow control insert can improve flow distribution in the opposite direction through the sorbent cartridge, which may be useful in certain processes, such as recharging the sorbent material after use.

Other shapes for flow channels that approximate the shape of a cylinder, such as circular flow channels, can be used. In certain embodiments, the flow channels can be a set of non-concentric cylinders, as in a bundle of straws. However, the hexagonal and concentric cylinder flow channels illustrated in FIGS. 1-6 advantageously cover the entire surface of the flow control insert efficiently, allowing fluid to only travel through the flow channels without leaving excess dead space in the sorbent cartridge.

Although the hexagonal flow channels illustrated in FIGS. 1-4 and 6 are all shown as the same size, in certain embodiments the flow control inserts can have different sized flow channels. Different sized flow channels allow for the creation of unique flow distribution patterns through the sorbent cartridge. Having varying hexagon sizes with a flow control insert can result in a desired flow distribution. For example, smaller hexagons can be used towards the center of the flow control insert, with the hexagons increasing in size towards approach the sides of the sorbent cartridge. Smaller hexagons towards the center and larger hexagons towards the outside may prevent a parabolic flow pattern with faster flow at the center and slower flow towards the walls, which is common for flow through a cylinder without a flow control insert.

In certain embodiments, the flow channels can be filled with different sorbent materials. For example, certain flow channels can be filled with a first type of ion exchange resin and other flow channels can be filled with a second type of ion exchange resin to tailor solute removal from a fluid. Having different sorbent materials in different flow channels may result in specified partial solute removal. For example, if half of the hexagons are filled with cation exchange material such as zirconium phosphate and the other half of the hexagons are filled with an anion exchange material such as zirconium oxide, the sorbent cartridge would only remove half of the cations (Ca⁺, Mg⁺, K⁺) and half of the anions (PO4⁻). In some cases, it may be beneficial to remove only a portion of the phosphate or cations from a patient during dialysis. Phosphate in particular is a common ion that can be over removed during traditional dialysis that happens more than 3 times per week. It is common for phosphate to be added as an infusate in the dialysate, however, if only a portion of the flow channels have an anion exchange material, less of the phosphate will be removed, reducing the need to add additional phosphate to the dialysate. In certain embodiments, a portion of the flow channels can be kept empty to tailor a particular % removal of a solute from the dialysate.

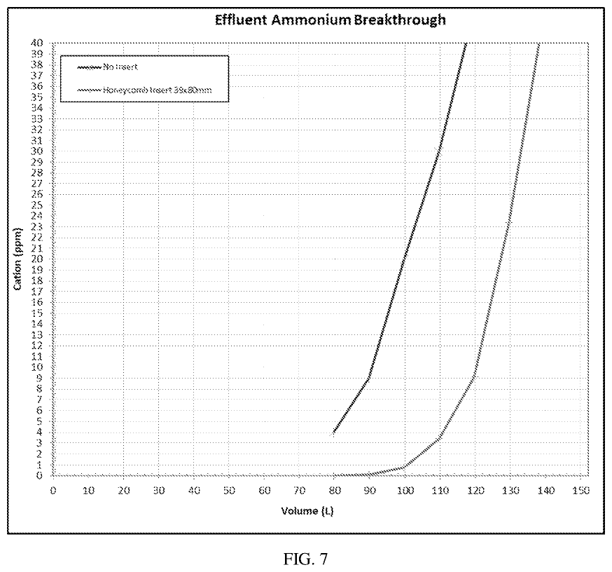

As described, by improving the flow distribution through the sorbent cartridge, the efficiency of solute removal by the sorbent cartridge is increased. FIG. 7 is a graph showing the volume of dialysate that can be flowed through a zirconium phosphate sorbent cartridge before breakthrough occurs. The x-axis in FIG. 7 is the volume of fluid pumped through the sorbent cartridge. The y-axis in FIG. 7 is the concentration of ammonium ions in fluid exiting the sorbent cartridge. To test the sorbent cartridges, a simulated spent dialysate feed having 130 mM Na, 36 mM, HCO₃, 3.2 mM K, 2 mM Ca, 1 mM Mg, 23.4 mM NH₄, plus other components was pumped through each sorbent cartridge at 600 ml/min at a temperature of 37° C. The higher line in FIG. 7 shows the ammonium ion concentration vs. volume for a zirconium phosphate sorbent cartridge without a flow control insert, while the lower line shows the ammonium ion concentration vs. volume for a zirconium phosphate sorbent cartridge with a flow control insert similar to that illustrated in FIG. 3A. As illustrated in FIG. 7, significant levels of ammonium ions were present in fluid exiting the sorbent cartridge without the flow control insert by the time 80 L of simulated spent dialysate was pumped through the sorbent cartridge. In contrast, when using the flow control insert, significantly more volume can be treated by the sorbent cartridge before reaching similar levels of cations in the treated fluid. Using the flow control insert, the ammonium capacity at 10 ppm increases from about 90 L without the flow control insert to about 120 L with the flow control insert, which corresponds to about a 33% in capacity.

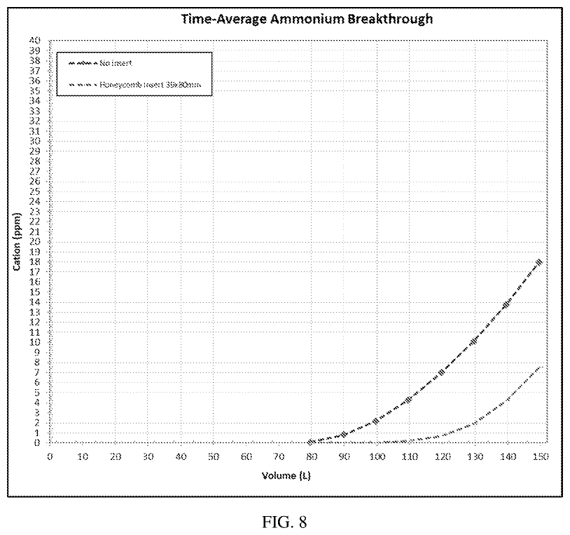

FIG. 8 is a graph showing the time-averaged effluent level of ammonium ions vs. volume of simulated spent dialysate passed through the cartridge. The higher line in FIG. 8 is the time averaged ammonium ion concentration vs. volume for a zirconium phosphate sorbent cartridge without a flow control insert, while the lower line shows the time averaged ammonium ion concentration vs. volume for a zirconium phosphate sorbent cartridge with a flow control insert similar to that illustrated in FIG. 3A. The simulated spent dialysate and conditions used for the data of FIG. 8 are the same as described with respect to FIG. 7. As illustrated in FIG. 8, the time-average ammonium capacity at 2-ppm increases from 100 liters without using a flow control insert to 130 liters with a flow control insert, a 30% increase.

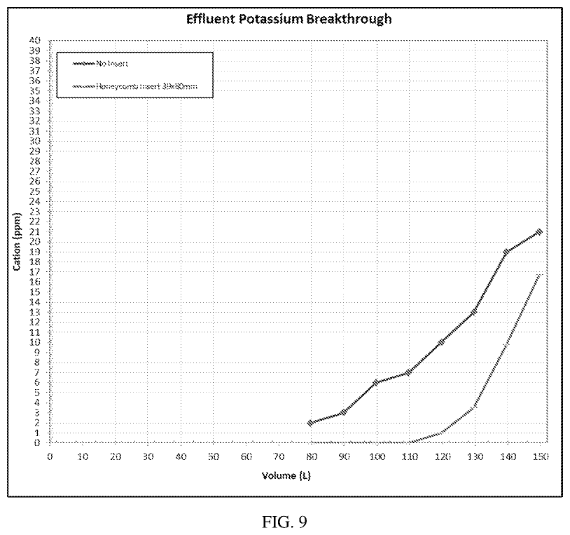

FIG. 9 is a graph showing the instantaneous potassium concentration in fluid exiting a sorbent cartridge vs. volume of dialysate pumped through the sorbent cartridge. The data in FIG. 9 was obtained with the same conditions and materials as that in FIGS. 7-8. The higher line in FIG. 9 is the instantaneous potassium ion concentration vs. volume for a zirconium phosphate sorbent cartridge without a flow control insert, while the lower line shows the instantaneous potassium ion concentration vs. volume for a zirconium phosphate sorbent cartridge with a flow control insert similar to that illustrated in FIG. 3A. As illustrated in FIG. 9, the sorbent cartridge potassium capacity at 8-ppm increases from 112 liters without a flow control insert to 137 liters when using the flow control insert, a 22% increase.

As illustrated in the graphs of FIGS. 7-9, the flow control insert significantly increases the capacity of a sorbent cartridge by improving fluid flow distribution through the sorbent cartridge.

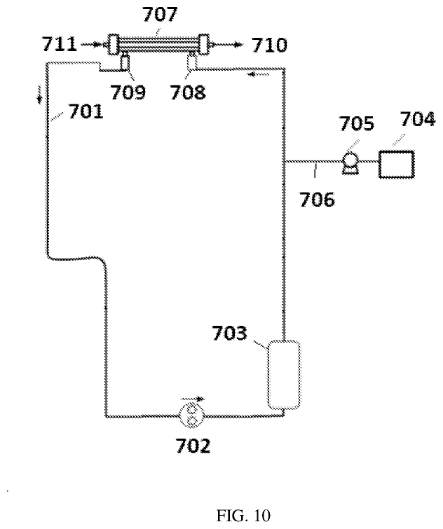

FIG. 10 illustrates a simplified dialysate flow path 701 for use in hemodialysis. Spent dialysate can be pumped through a dialyzer 707 from a first connector 708 fluidly connected to the inlet of the dialyzer 707 to a second connector 709 fluidly connected to an outlet of the dialyzer 707. At the same time, blood from a patient enters the dialyzer 707 through blood inlet 711 and exits through blood outlet 710. Dialysate pump 702 provides the force necessary for moving dialysate through the dialysate flow path 701. The dialysate exiting dialyzer 707 can be regenerated by passing the dialysate through a sorbent cartridge 703. As described herein, the sorbent cartridge 703 can contain a flow control insert, along with one or more sorbent materials. The sorbent materials in sorbent cartridge 703 can include zirconium phosphate for removal of cations from the dialysate, zirconium oxide for removal of anions from the dialysate, and activated carbon for removal of creatinine, glucose, uric acid, β2-microglobulin and other non-ionic toxins, except urea, from the dialysate. The sorbent cartridge can also include urease, which converts urea to ammonium ions and carbon dioxide with the ammonium ions removed by the zirconium phosphate. In certain embodiments, the urease can be immobilized on alumina to prevent the urease from escaping the sorbent cartridge. Because the zirconium phosphate will remove potassium, calcium, and magnesium from the spent dialysate, cations can be added back into the dialysate from infusate source 704. The infusate solution can be added through fluid line 706 by infusate pump 705. Although shown as a single infusate source 704 in FIG. 10, one of skill in the art will understand that the system can include any number of infusate sources. Additional components, such as a sodium source, a bicarbonate source, a water source, a degasser and any other components of a dialysis system can be added.

Although shown as a single sorbent cartridge 703 in FIG. 10, the sorbent materials can be contained within any number of sorbent modules. For example, a first sorbent module can contain activated carbon, urease, and alumina, while a second sorbent module contains zirconium phosphate, and a third sorbent module contains zirconium oxide. Each of the sorbent modules can contain a flow control insert. Separating the sorbent materials into different modules can ease recharging of the individual sorbent materials without the need to remove the sorbent materials from the sorbent cartridge. Any number of sorbent modules can be used containing any combination of sorbent materials, so long as zirconium phosphate is located downstream of any urease.

One skilled in the art will understand that various combinations and/or modifications and variations can be made in the described systems and methods depending upon the specific needs for operation. Various aspects disclosed herein may be combined in different combinations than the combinations specifically presented in the description and accompanying drawings. Moreover, features illustrated or described as being part of an aspect of the disclosure may be used in the aspect of the disclosure, either alone or in combination, or follow a preferred arrangement of one or more of the described elements. Depending on the example, certain acts or events of any of the processes or methods described herein may be performed in a different sequence, may be added, merged, or left out altogether (e.g., certain described acts or events may not be necessary to carry out the techniques). In addition, while certain aspects of this disclosure are described as performed by a single module or unit for purposes of clarity, the techniques of this disclosure may be performed by a combination of units or modules associated with, for example, a medical device.

What is claimed is:

1. A dialysate system comprising:
a first sorbent module located in a dialysate flow path,
wherein the first sorbent module comprises:
a flow control insert comprising a plurality of flow channels,
wherein the plurality of flow channels is configured to be filled with at least one sorbent material;
wherein the flow control insert is configured to distribute fluid flow more efficiently throughout the at least one sorbent material to increase an adsorptive capacity of the first sorbent module.

2. The dialysate system of claim 1, wherein the at least one sorbent material comprises at least one of activated carbon, alumina, urease, zirconium phosphate, zirconium oxide, anion exchange material, cation exchange material, or any combination thereof.

3. The dialysate system of claim 1, wherein the at least one sorbent material comprises zirconium phosphate.

4. The dialysate system of claim 1, wherein the at least one sorbent material comprises zirconium oxide.

5. The dialysate system of claim 1, wherein each of the plurality of flow channels has a hexagonal shape.

6. The dialysate system of claim 1, wherein the plurality of flow channels form a plurality of cylinders.

7. The dialysate system of claim 6, wherein the plurality of cylinders is a plurality of concentric cylinders.

8. The dialysate system of claim 1, wherein the flow control insert has a substantially flat top portion.

9. The dialysate system of claim 1, wherein the flow control insert has a dome-shaped top portion.

10. The dialysate system of claim 1, wherein the flow control insert is constructed from at least one of stainless steel, glass, plastic, or any combination thereof.

11. The dialysate system of claim 1, wherein the flow control insert is constructed from at least one of polypropylene, polyethylene, or any combination thereof.

12. The dialysate system of claim 1, wherein at least one of the plurality of flow channels is filled with the at least one sorbent material.

13. The dialysate system of claim 12, wherein the at least one sorbent material comprises at least one of activated carbon, alumina, urease, zirconium phosphate, zirconium oxide, anion exchange material, cation exchange material, or any combination thereof.

14. The dialysate system of claim 1, wherein the plurality of flow channels are at least partially filled with the at least one sorbent material.

15. The dialysate system of claim 14, wherein the at least one sorbent material comprises at least one of activated carbon, alumina, urease, zirconium phosphate, zirconium oxide, anion exchange material, cation exchange material, or any combination thereof.

16. The dialysate system of claim 1, further comprising:
a second sorbent module located in the dialysate flow path.

17. The dialysate system of claim 16, wherein the second sorbent module comprises:
a flow control insert comprising a plurality of flow channels,
wherein the plurality of flow channels is configured to be filled with at least one sorbent material;
wherein the flow control insert is configured to distribute fluid flow more efficiently throughout the at least one sorbent material to increase an adsorptive capacity of the second sorbent module.

18. The dialysate system of claim 17, wherein the at least one sorbent material of the second sorbent module comprises at least one of activated carbon, alumina, urease, zirconium phosphate, zirconium oxide, anion exchange material, cation exchange material, or any combination thereof.

19. The dialysate system of claim 17, wherein each of the plurality of flow channels of the flow control insert of the second sorbent module has a hexagonal shape.

20. The dialysate system of claim 17, wherein the at least one sorbent material of the second sorbent module is different from the at least one sorbent material of the first sorbent module.
